(19) United States
(12) Patent Application Publication
(10) Pub. No.: US 2008/0300558 A1
(43) Pub. Date: Dec. 4, 2008

(54) Article

(75) Inventors: Ulla Forsgren Brusk, Pixbo (SE); Chatrine Stridfeldt, Hovas (SE); Bo Runeman, Partille (SE); Carolyn Berland, Molndal (SE)

Correspondence Address: DRINKER BIDDLE & REATH (DC), 1500 K STREET, N.W., SUITE 1100, WASHINGTON, DC, 20005-1209, US

(73) Assignee: SCA HYGIENE PRODUCTS AB, Goteborg (SE)

(21) Appl. No.: 12/158,023

(22) PCT Filed: Dec. 20, 2005
(86) PCT No.: PCT/SE2005/001970

§ 371 Date: Jun. 18, 2008

(51) Int. Cl. A61F 13/15

(52) U.S. Cl. 604/360; 604/367; 604/385.03; 604/385.23; 604/389

(57) ABSTRACT

An absorbent article for female use, which prevents or reduces migration of microorganisms from the anus area to the urogenital area during use. The article includes at least one first microorganism hindering element or section having a front border, a rear border and two side borders, the first element or section being situated so that it will be placed against the perineum of the wearer during use for preventing or reducing migration of microorganisms in the direction from the rear edge to the front edge of the article, the first element being arranged on the top sheet or the section being incorporated in the top sheet.

### CROSS-REFERENCE TO PRIOR APPLICATION

This application claims priority to Application No. PCT/SE2005/001970 filed Dec. 20, 2005, which is incorporated by reference herein.

### FIELD OF THE INVENTION

The present invention relates to absorbent articles and microorganism hindering articles for female use, including a microorganism hindering element or section for preventing urogenital infections.

### BACKGROUND OF THE INVENTION

Infections in the urogenital region are a problem that affects many individuals. In and around the anus, there are many kinds of microorganisms. It is known that one reason for many infections in the urogenital region is that microorganisms from a person's own intestinal flora spread from the anus via the perineum to the urogenital area and there cause infection.

Normally, an ecological balance prevails between different microorganisms on skin and mucus membranes, and the normal microbiological flora is highly significant in preventing the establishment of undesirable microorganisms. However, this natural defense system is sometimes inadequate and can be disturbed in a way that enables potentially pathogenic microorganisms to become established and give rise to infection, for instance in conjunction with medication, poor hygiene, skin changes, changes in mucus membranes and long term use of absorbent articles.

Regarding the danger of contracting infections in the urogenital region, women are more at risk than men due to the short distance from the anus to the urethral orifice and vagina. Prolonged use of articles without regular changing or poor genital hygiene combined with impaired immunological defenses might substantially increase the risk for the spread of microorganisms. Further, tight clothing and underwear might increase the risk of migration of microorganisms. The microorganism density in most women is within normal levels that are tolerable in a healthy woman's vulvar environment. The densities are in most cases low for potentially pathogenic species, like S. aureus, E. coli and C. albicans. However, if unwanted species are present, even in low numbers, there is a possibility that in some situations the microorganisms might grow in number.

A natural part of the prophylaxis against infections in the urogenital area is enhanced personal hygiene. However, it can be inappropriate to wash the genitals and lower abdomen with a strong soap or bactericidal substances, and consequently it may be difficult for an individual to reduce the risk of infection to a sufficient level with the aid of conventional means. It may be difficult at times for women to change feminine articles. Long term use of feminine articles may also increase the risk for infections due to microorgansims accumulated in the article. Occlusion and temperature may promote the growth of microorganisms in the article and on the skin and mucus membrane of the user.

Traditionally, the aforesaid problems have been addressed by treating resultant infections with conventional antibiotics. However, frequent treatment with antibiotics can lead to the development of resistant bacteria strains, which can make continued treatment of new infections very difficult. A further problem with antibiotic treatment is that many individuals are hypersensitive to antibiotics. Further, antibiotics are harmful also to the beneficial skin and vaginal flora which makes reinfection more likely.

### DESCRIPTION OF THE BACKGROUND ART

As mentioned above, a traditional method of dealing with the aforesaid problems is to treat the patient with conventional antibiotics.

It has also been proposed to use lactic acid bacteria because of their inhibiting effect on pathogens. Application of such Lactic acid bacteria has been found to reduce the occurrence of infections on both skin and mucus membranes.

WO 2004/105822 describes for example the use of absorbent articles provided with lactic acid producing bacteria. The bacterial cells are transferred to the skin of a user and prevent and treat microbial infection in the urogenital area and on the skin.

EP 1 032 434 B1 discloses an absorbent product which contains lactic acid bacteria; the bacteria are arranged to be transferred to the user's skin in order to impair the living conditions for undesirable strains of microorganisms.

Further, US 2004/0192642 A1 teaches a composition for treating and preventing urogenital infections. The composition is administered to a mammalian vagina by a syringe-like applicator or by a tampon.

WO 97/02846 further discloses a tampon with added microorgansisms for preventing urinary tract infections.

The use of tampons or applicators to be inserted in vagina, for preventing urogenital infections, could be considered cumbersome by some individuals. Lactic acid bacteria require special protection during storage. Therefore a more stable solution to the problem is preferable.

As discussed in the introduction, any unwanted species present in the urogenital region even at low numbers, create the possibility that pathogenic microorganisms might grow in number in some situations. Thus, there is a need for microorganism hindering articles to prevent microorganisms from migrating from the anus to the urogenital area. The present invention intends to solve these problems.

### SUMMARY OF THE INVENTION

The object with the present invention is to provide an absorbent article for female use, such as a sanitary napkin, a panty liner or an incontinence guard, or a microorganism hindering article for feminine use, which prophylactically reduces the risk for urogenital infections by preventing or reducing migration of microorganisms from the anus area to the urogenital area.

This is achieved by the present invention by an absorbent article for female use, which includes at least one first microorganism hindering element or section, for preventing or reducing migration of microorganisms from the anus area to the urogenital area in the direction from the rear edge to the front edge of the article.

The present invention also refers to a microorganism hindering article for female use, which includes at least one first microorganism hindering element or section, for preventing or reducing migration of microorganisms from the anus area to the urogenital area in the direction from the rear edge to the front edge of the article.

Further, the present invention relates to a kit including a) an absorbent article, such as a sanitary napkin, a panty liner or an incontinence guard, and b) a microorganism hindering article including a first microorganism hindering element or section.

Moreover, the invention relates to a kit including a) an absorbent article, such as a sanitary napkin, a panty liner or an incontinence guard, having a marking on the top sheet, b) a composition for preventing or reducing migration of microorganism and c) a means for applying the composition on the marking on the top sheet.

The absorbent article and the microorganism hindering article are intended to be used in such a way so that the first element or section is positioned against the perineum of the wearer during use and thereby the migration of microorgansims from the anus area to the urogenital area, via the skin of the perineum, is prevented or reduced.

### BRIEF DESCRIPTION OF THE DRAWINGS

FIG. 1 discloses an anatomic cross-sectional view of a human female illustrating the genitals.

FIG. 2 discloses a top plan view of an absorbent article, in the form of sanitary napkin, according to the invention.

FIG. 3 discloses a top plan view of a microorganism hindering article according to the invention.

FIG. 4 discloses a top plan view of an alternative embodiment of a sanitary napkin according to the invention.

FIG. 5 discloses a cross-section of an absorbent article according to an embodiment of the invention and a cross-sectional view of a human female illustrating the genitals.

FIG. 6 discloses a cross-section of an absorbent article according to a further embodiment of the invention.

FIG. 7 discloses a cross-section of a sanitary napkin according to an embodiment of the invention.

FIG. 8 discloses a top plan view of a sanitary napkin according to the invention.

FIG. 9 discloses a top plan view of a panty liner according to the invention.

FIG. 10 discloses a top plan view of a sanitary napkin according to the invention having a stiffening element.

For features indicated with a three digit reference number, the same last two digits have been used for like parts, but the first digit differs from figure to figure. Reference numbers are not shown in all figures, where the corresponding parts can be found in another figure.

### DETAILED DESCRIPTION OF THE INVENTION

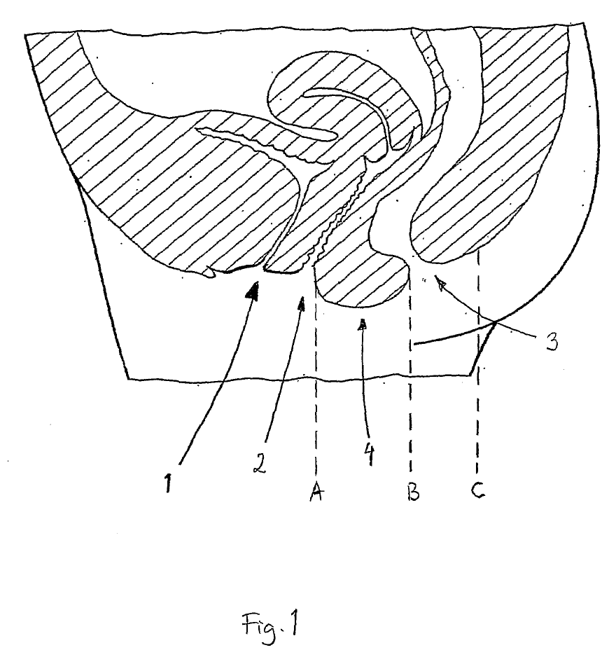

In order to further explain the invention, reference is made to FIG. 1. FIG. 1 shows an anatomic cross-sectional view of a human female illustrating the genitals. The external urethral orifice 1, the vagina 2 and the anus 3 are shown. The perineum 4 is situated between the anus 3 and the vagina 2. Points A and B are shown, which correspond to the front and rear part of the perineum area, respectively. The distance between the anus 3 and the vagina 2, i.e., between A and B, is about 1 to 4 cm in women. The microorganisms from the anus area can migrate from the anus 3 over the perineum 4, i.e., from B to A, and there after reach the urogenital area, i.e., the vagina 2 and the urethra orifice 1. Such a migration of microorganisms can, under certain circumstances, cause urinary tract infection and/or vaginal infections. The purpose with the present invention is to reduce the risk of infection by avoiding such a migration through hindering the microorganisms from migrating from the anus 3 to the urogenital area over the perineum 4. This is achieved by an absorbent article or a microorgansim hindering article, which are adapted to fit female anatomy.

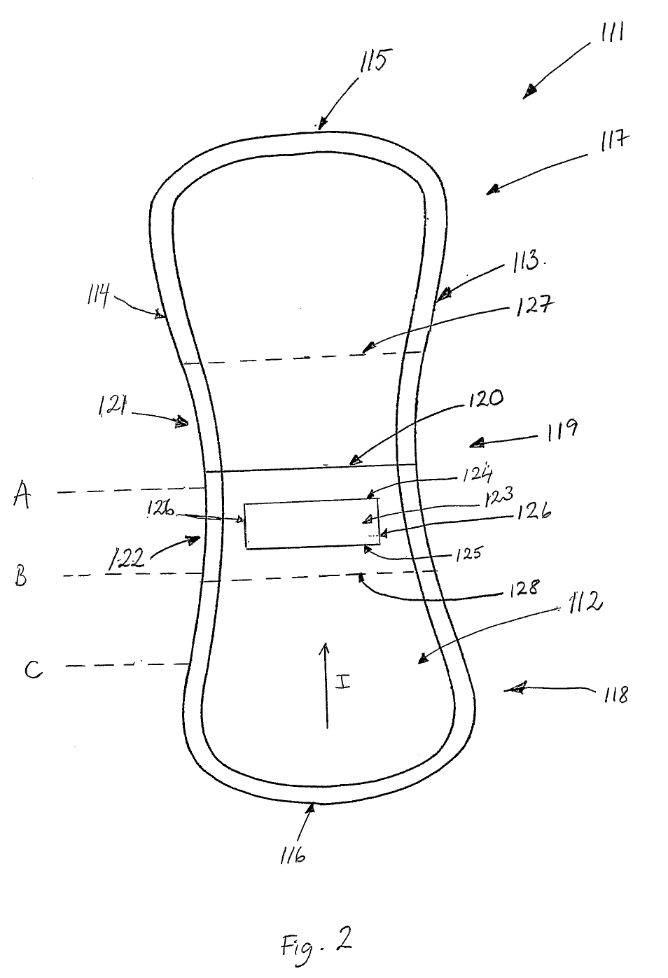

The present invention refers to an absorbent article 111 for female use, such as a sanitary napkin, a panty liner or an incontinence guard, wherein a sanitary napkin is shown in FIG. 2, which prevents or reduces migration of microorganisms from the anus area to the urogenital area during use, including a liquid-pervious top sheet 112 intended to be directed against the wearer during use and a liquid-impervious bottom sheet (not shown) intended to be directed against the underpants of the user during use, which article has a longitudinal direction and a transverse direction, two essentially longitudinal side edges 113, 114, one essentially transverse front edge 115, one essentially transverse rear edge 116, a front portion 117, a rear portion 118, and a crotch portion 119 located between the rear portion 118 and the front portion 117, the crotch portion 119 being divided into a front part 121 and a rear part 122 by a central line 120, the crotch portion 119 having a length of about 7 to 12 cm, wherein the article includes at least one first microorganism hindering element 123 or section, having a front border 124, a rear 125 border and two side borders 126, the first element 123 or section being situated so that it will be placed against the perineum of the wearer during use for preventing or reducing migration of microorganisms in the direction from the rear edge 116 to the front edge 115 of the article, the first element 123 being arranged on the top sheet 112 or the first section being incorporated in the top sheet 112. Further, the first element 123 or section is arranged so that the front border 124 of the first element 123 or section is situated in the rear part 122 of the crotch portion 119. According to an embodiment the front border 124 of the first element 123 or section is situated about 0.5 to 3 cm behind the center line 120 of the crotch portion 119. The first element 123 is shown in the Figure. The section is situated on the same place, when looking at the Figure from above.

For illustrating the crotch portion 119, a front border 127 and a rear border 128 of the crotch portion 119 are shown with broken lines. Further, the crotch portion 119 is divided by a center line 120 in order to show the limit between the front part 121 and rear part 122 of the crotch portion 119. The center line 120 is placed essentially in the middle of the crotch portion 119. The placement of the first element 123 or section on the article depends on how the product will be positioned against the wearer. The positioning on the wearer differs depending on what product is concerned. However, the crotch portion is always intended to be placed against the crotch of the user and the first element or section should always be placed against the perineum. Further, the points A, B and C are also marked, corresponding to the points in FIG. 1. This shows that the first element or section is intended to be positioned between A and B, i.e., on the perineum.

The absorbent article according to the present invention can further include an absorbent layer between the top sheet and the bottom sheet. The absorbent layer is preferably constructed from cellulose fibers, although other natural materials may be used, such as cotton fibers or peat. Alternatively, absorbent synthetic fibers or a mixture of natural fibers and synthetic fibers may be used. The absorbent layer may also include an open cell foam. The absorbent layer may also include a superabsorbent, i.e., a polymer that is able to absorb liquid in an amount corresponding to several times its own weight. Further, the absorbent layer may contain odor-inhibiting additives, such as zeolites, silica, active coal and lactobacilli.

Conventional materials are used in the bottom sheet and the top sheet of the absorbent article.

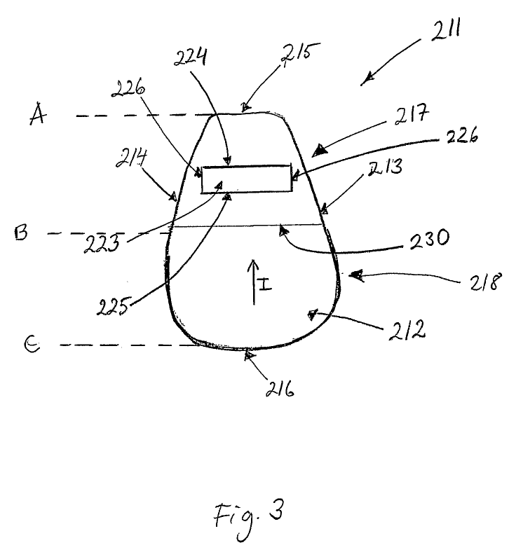

Further, the present invention relates to a microorganism hindering article 211 for feminine use, shown in FIG. 3, for preventing or reducing migration of microorganisms from the anus area to the urogenital area during use, including a top sheet 212 intended to be directed against the wearer during use and optionally a liquid-impervious bottom sheet (not shown) intended to be directed against a user's underpants during use, which article 211 has a longitudinal direction and a transversal direction, two essentially longitudinal side edges 213, 214, one essentially transversal front edge 215, one essentially transversal rear edge 216, a front portion 217, and a rear portion 218, wherein it includes at least one first microorganism hindering element 223 or section, having a front border 224 and a rear 225 border and two side borders 226, the first element 223 or section being intended to be placed against the perineum of the wearer during use for preventing or reducing migration of microorganisms in the direction from the rear edge 216 to the front edge 215 of the article, the first element 223 being arranged on the top sheet 212 or the first section being incorporated in the top sheet 212. The front part 217 and the rear part 218 are divided by a central line 230. According to an embodiment, the first element 223 or section is situated in the front portion 217 of the article.

Limits A, B and C are also shown in FIG. 3 and they correspond to the points A, B and C in FIG. 1. These are used for illustrating how the microorganism hindering article should be placed against the wearer so that the first element 223 or section will be aligned with the perineum 4. The microorganism hindering article 211 according to the present invention should be placed with the transverse front edge 215 just behind the vagina 2, at point A. The first element 223 or section will then be placed against the perineum thereby hindering microorganisms from passing from the anus towards the urogenital area, i.e., towards the vagina 2 and the urethra 1, in the direction from the rear edge 216 to the front edge 215 of the article. The limit C is also shown on the article in FIG. 3, but it is not necessary that the article extends to the anus area. The microorganism hindering article is designed to be easy for women to use, they can apply the article against the perineum by feeling how to place the article just behind the vagina 2. Thus, despite the novelty of this new article for feminine use it will be easy to use in practice. It is small and should only be positioned in the perineum area, it could optionally also have an extension behind the perineum.

The top sheet 212 preferably includes a soft, skin-friendly material. This top sheet 212 may include a sheet of different types of nonwoven material. Alternative materials in this respect are perforated plastic films, plastic nets, knitted, crocheted, low density air-laid, tissue, woven or foam materials, and combinations and laminates of these types of materials. The plastic may be a thermoplastic, e.g. polyethylene (PE). The nonwoven material may include natural fibers, such as cellulose or cotton, although it may alternatively include synthetic fibers, such as polyethylene (PE), polypropylene (PP), polyurethane (PU), a polyester, nylon or regenerated cellulose, or a mixture of different fibers. All materials that are used to provide liquid-permeable outer sheets in absorbent articles, such as sanitary napkins, panty liners or incontinence protectors, can be used for the top sheet 212, and it will be understood that the aforesaid materials are given merely by way of example. These materials may also be used for the top sheet 112 in the absorbent article according to the present invention.

The liquid-impermeable bottom sheet may include a flexible material, preferably a thin film of polyethylene (PE), polypropylene (PP) or polyester, although it may also include a lamination of nonwoven material with liquid impermeable material. All materials that are typically used to produce liquid-impervious bottom sheets for absorbent articles can be used. The bottom sheet may conveniently be air-permeable. Further, these materials may also be used in the bottom sheet of the absorbent article.

In the microorganism hindering article, the top sheet may be of the kind used in absorbent articles, and also the bottom sheet could be according to conventional bottom sheets used in absorbent articles.

The distance between the transverse edges 215, 216 is from 2 cm to 6 cm and the distance between the longitudinal edges 213, 214 is from 3 cm to 5 cm in the microorganism hindering article. In the microorganism hindering article in FIG. 3, the front transverse edge 215 is shorter than the rear transverse edge 216. This design is adapted to the female anatomy. It is however, not necessary that the article has this form. If the article is symmetric, it will preferably indicate in a simple and clear way what part of the article is the front or rear in order to allow a user to align the article in the correct way on the perineum. The first hindering element or section could be shown on the top sheet by a marking or coloring so it is clear how to use the article.

The use of a microorganism hindering article or an absorbent article according to the present invention has the effect that it hinders microorganisms from migrating from the anus 3 to the urogenital area, when the article is placed so that the first element or section is situated against the perineum of the wearer. Through close physical contact between the article and the skin of the perineum, a microorganism hindering barrier will be formed on the skin of the perineum. Thus, the first element or section will prevent or reduce migration of microorganisms from the anus region to the urogenital region in the direction from the rear edge to the front edge of the article. The barrier to microorganism transport can be formed either by close contact between the first element or section of the article and the skin of the perineum or by transferring some material from the first element or section to the skin of the perineum thereby producing a barrier on the skin of the perineum. The migration of microorganisms from anus to the urogenital area is thereby reduced or prevented. This will be described further below.

When the expression “article” is used alone, it is intended to encompass both the microorganism hindering article and the absorbent article. In some specific embodiments, it is clarified which particular article is being described. The first element or section is designed in the same way in both the microorganism hindering article and the absorbent article but the articles themselves are designed differently.

According to the present invention, the first element or section includes a material selected from one or more of a hydrophobic material, an antimicrobial agent and a positively charged material.

In one embodiment of the invention, the first element or section includes a hydrophobic material. Such a material could for example be coated on the top sheet or incorporated in the top sheet. If the material is incorporated, the hindering material is considered to be included in a section of the top sheet. If the hydrophobic material is applied on the top sheet, the material is considered to be included in an element arranged on the top sheet.

When a hydrophobic material is used in the first element or section, it could be transferred to the skin of the wearer and could thus act on both the skin and the article to hinder migration of microorganisms. The hydrophobic material should be chosen so that the material can be transferred to the skin of the wearer in a suitable way. The hydrophobic material could be semisolid at about 30 to 32° C. Further, it could be semisolid up to about 34° C. When the material is semisolid at ambient temperature its migration is minimized when the article is not in use. It is important that the material does not migrate on the top sheet, since liquid intake through the top sheet could be reduced or prevented by the hydrophobic material. It is also important that the material does not migrate further down in an absorbent article, since the absorption capacity could be negatively affected. It is of special importance that there is a close contact between the first element or section and the skin of the wearer or that material from the first element or section is transferred to the skin so that the migration of microorgansims from the anus area to the urogenital area is prevented or reduced.

It is important that the material is transferred to the skin, but it should not flow on the skin or on the article. Fluid materials could move to unintended places, flow off the wearer, or even spread out on the article, the latter of which is a disadvantage regarding liquid inlet of the top sheet and absorbance capacity of an absorbent layer. Therefore, the material should have an appropriate viscosity so it will not be smeared out. Suitable viscosities are 5-15 PaS at 35° C. The method for measuring this is made at a ramp rate of 1° C./min, a shear rate of 9.25 s⁻¹ and with a measurement interval of 20-50° C.

A hydrophobic material has a wetting angle above 90° and should be substantially free from water. The material can be selected from lipids, fatty alcohols, fatty acid esters, polyalcohols, complex lipids, waxes and polysiloxanes, and mixtures thereof. The lipids could for example be paraffins or triglycerides. Examples of paraffins are paraffin oil or petrolatum. Examples of triglycerides are refined and/or hydrated triglycerids from animal or plant origin, with preferably carbon chain lengths of under C-18 (e.g., milk fat, coconut oil Cocous nocifera, palm-kernel oil Elaeis guineeis), animal or vegetable with unsaturated C-18 fatty acids (e.g., Japan wax Rhus succesdanes, tallow fat, soybean oil Glycerin soya, peanut oil Arachais hypogaea, maize oil Zea mays, sunflower oil Helanthus annus, grapeseed oil Vitis vinifera, safflower oil Carthamus tinctorius, sweet almond oil Prunnus amygdalus dulcis, hazelnut oil Corylus americana, walnut oil Juglans regia, olive oil Olea europasa, avocado oil Persea gratissima, sesame oil Sesamum indicum, tall oil, Tallol, cottonseed oil Gopssypium, palm oil Elaesis guineensis, rice oil Oryza sativa, rape oil Canola, apricot-kernel oil Prunus armeniaca, cocoa butter Theobroma cao, shea butter Butyrospermum parkii, whatseed oil Triticum vulgare, Bassia latifola), or animal or vegetable with carbon chains over C-18 (e.g., beeswax Cera alba, shellac wax Shellac cera, meadowfoam seed oil Limnanthes alba, rapeseed oil Brassica capmestris, cucumberseed oil Borago officinalis, linseed oil Linum usitatissimum, ricin oil Ricinus communis, veronia oil Veronia galamensis, jojoba oil Buxus chinensis, candlewax Euphorbia cera, ongokea oil Ongokea gore).

Fatty alcohols could be selected among straight or branched carbon chain lengths of 12-32 carbons. Examples are cetyl alcohol or stearyl alcohol. The fatty acid esters could have 12-32 carbons and could for example be selected among methyl palmitate, methyl stearate, isopropyl myristate, isopropyl laurate, isopropyl palmitate, isopropyl stearate, octyl palmitate, octyl stearate or octyl laurate. The polyalchols could be selected from sugar alcohols or polyglycerols for example. Examples of complex lipids are phospholipids or sphingolipids (ceramides). The waxes can be of animal origin and for example selected from beeswax or lanolin. They could be of mineral origin and be selected from ozocerite or ceresin. Further, they could be of plant origin, e.g., Carnauba or candelilla. The polysiloxanes could be straight, branched or cyclic and could for example be selected from polydimethylsiloxane (dimethicone) or polydiethylsiloxane.

The hydrophobic material can be applied in any suitable form. It can be applied by spraying, slot coating, or printing. Further, the material can be impregnated, or penetrated into the top sheet. The material could also be applied in the form of beads. As mentioned above, the material is included in a first element or section. The section is considered to be a part of the top sheet, while the element is considered to be a separate part applied on the top sheet. The section could be applied by melting the hydrophobic material down in the top sheet, or in some other suitable way.

Concentrations of the hydrophobic material can be 0.1 to 100 g/m² in the treated area or 10-100 g/m² in another embodiment according to the present invention.

The microorganisms from the anus will not be able to move from the anus and pass the perineum, when the first element or section is in close contact with the skin of the wearer. As already mentioned, the migration of microorganisms is prevented on the article and especially on the skin of the wearer. The hydrophobic material will prevent or reduce microorganisms from moving from the anus region to the urogenital region, i.e., in a direction forward, which is indicated by an arrow I in FIGS. 2 and 3. The microorganisms can be prevented from moving both on the skin of the wearer and on the microorganism hindering article or only on the absorbent article or the microorganism hindering article or only on the skin during use. However, it is preferred that the migration is prevented or reduced on the skin of the perineum.

The reason why the hydrophobic material works as a hindering media for microrgansisms, such as bacteria, is believed to be that the bacteria will adhere to the hydrophobic material. Many bacteria, in particular many pathogenic bacteria, have hydrophobic areas on the cell wall and/or hydrophobic projections in the cell wall (pilli, fimbriace etc) and thus adhere strongly to hydrophobic surfaces. The bacteria will therefore not pass the first element or section situated on the perineum during use. Bacteria from the anus area can normally extend towards the perineum. When using the article according to the invention, a substantial number of the bacteria moving toward the urogenital area may reach the first element or section and will there be caught by the material in the first microorgansim hindering element or section. The bacteria can also be caught by the material from the first element or section, which has been transferred to the skin of the wearer.

According to another embodiment, the first element or section includes an antimicrobial agent. The antimicrobial agent can be selected from chitosan, metal oxides, metal or metal salts like silver and silver salts, copper and copper salts, copper acetate, quaternary ammonium salts, chlorhexidine, parabens (methyl, ethyl and propyl), pH-buffered materials, disodium ethylenediaminetetraacetic acid (EDTA), and other chelating agents, chlorhexidine digluconate, amorolfin, bacitracin, benzalkonium chloride, benzetonium chloride, cetrimide, fusidic acid, gentian violet (methylrosaniline chloride), hexachlorophene, hexylresorcinol, imidazole derivatives (for example biphonazole, econazole, ketoconazole, chlotrimazole, miconazole), nystatin, povidone-iodine, elemental iodine and iodophores, terbinafin, triclosan, hydrogen peroxide, or halogenated phenylene compounds. The antimicrobial agent could also be an acidic material, i.e a material having a low pH, also called a pH control agent. pH control agents can be selected from citric acid, adipic acid, azelaic acid, malic acid, tartaric acid, gluconic acid, glutaric acid, levulenic acid, glycolic acid, succinic acid, fumaric acid, pimelic acid, suberic acid, sebacic acid, sodium citrate and acid phosphate salts, carboxymethylcellulose, oxidized cellulose, sulfoethylcellulosa and phoxyphorylated cellulose. The antimicrobial agent can be coated on the top sheet or incorporated in the top sheet or applied on the top sheet in any suitable way. The agent could for example be sprayed, glued, printed, coated, such as slotcoated, on the top sheet or into the top sheet, if it is in such a condition so that the agent can penetrate into the top sheet. It could also be applied by use of fibers or films incorporating the agent. As with the hydrophobic material, it is important to have close contact between the first element or section and the skin of the user for elements or sections including the antimicrobial agent. The agent could also be transferred to the skin in the same way as the hydrophobic agent and thereby act on the skin of the wearer. The appropriate concentration needed to hinder bacterial transmission will be strongly dependent on the nature of the antimicrobial agent. It may be in the amount from ppm to grams. A person skilled in the art will be able to select an appropriate amount to achieve the desired antimicrobial effect.

Antimicrobial agents may kill many kinds of microorganisms. Therefore it is crucial that the antimicrobial agent and consequently the first element or section does not contact the genitals or urethra and thereby does not disturb normal flora in these areas.

Acidic antimicrobial agents can be activated by moisture, which will be present in an absorbent article or a microorganism hindering article after some time, such as sweat, menses, urine or other body exudates.

Microorgansims moving from the anus area towards the urogenital area will be inhibited and/or killed by the antimicrobial agent included in the first element or section. This will reduce the risk for urogenital infections, since the concentration of unwanted bacteria in the urogenital area will remain low.

The antimicrobial agent could be enclosed in a hydrophobic material. The hydrophobic material could be selected in order to protect the antimicrobial agent. In such a case, the hydrophobic material should be meltable, in order to release the antimicrobial agent. The melting can occur when the first element contacts the skin of the wearer. Thus, the melting temperature of the hydrophobic material would be less then 37° C. The hydrophobic material could also act both as an enclosing material and as the material transferring the antimicrobial agent to the skin of the wearer. In such a case, the hydrophobic material should have the same properties as the hydrophobic material used as a first element or section. It could be semisolid at about 30-32° C. Further, it could be semisolid up to about 34° C. Enclosing the antimicrobial agent could be useful if the antimicrobial agent needs protection for some reason before use. One reason could be that it is sensitive to moisture or air.

Another embodiment of the invention is an article wherein the first element or section includes a positively charged material for trapping microorgansims such as bacteria. Bacteria are negatively charged, so they will be attracted and bond to the positively charged material. Examples of positively charged materials are fibers which have been modified. Such fibers are disclosed in WO 2004/062703. Further examples are materials such as nonwoven or tissue treated with a positively charged polymer. Preferred polycationic polymers are preferably selected from homo- or copolymers of at least one monomer including a functional group that includes a nitrogen atom which can be protonated. They can have linear or branched structures.

Cationic polyelectrolytes can be selected from
a) cationic or cationically modified polysaccharides, such as cationic starch derivatives, cellulose derivatives, pectin, galactoglucommanan, chitin, chitosan or alginate;
b) a polyallylamine homo- or copolymer, optionally including modifier units (suitable modifier units of the polyallylamine are known for example from WO 00/31150), in particular polyallylamine hydrochloride (PAH);
c) polyethylenemine (PEI);
d) a polyvinylamine homo- or copolymer optionally including modifier units,
e) poly(vinylpyridine) or poly(vinylpyridinium salt) homo- or copolymer, including their N-alkyl derivatives,
f) polyvinylpyrrolidone homo- or copolymer, a polydiallyldialkyl, such as poly(N,N-diallyl-N,N-di-C₁-C₄-alkyl-ammonium halide) as shown in US 2004/0047979 A1, in particular poly(N,N-diallyl-N,N-dimethylammonium chloride) (PDDA);
g) a homo- or copolymer of a quaternized di-C₁-C₄-alkyl-aminoethyl acrylate or methacrylate, for example a poly(2-hydroxy-3-methacryloylpropyl-tri-C₁-C₂-alkylammonium salt) homopolymer such as a poly(2-hydroxy-3-methacryloylpropyl trimethylammonium chloride), or a quaternized poly(2-dimethylaminoethyl methacrylate or a quaternized poly(vinylpyrrolidone-co-2-dimethylaminoethyl methacrylate)
h) a poly(vinylbenzyl-tri-C₁-C₄-alkylammonium salt), for example a poly(vinylbenzyl-tri-methylammoniumchloride),
i) polymers formed by reaction between ditertiary amines or secondary amines and dihaloalkanes, including a polymer of an aliphatic or araliphatic dihalide and an aliphatic N,N,N′,N′-tetra-C₁-C₄-alkyl-alkylenediamine, for example a polymer of (a) propylene-1,3-dichloride or -dibromide or p-xylylene dichloride or dibromide and (b) N,N,N′,N′-tetramethyl-1,4-tetramethylene diamine,
j) POLYQUAD® as disclosed in EP-A-456,467; or
k) a polyaminoamide (PAMAM), for example a linear PAMAM or a PAMAM dendrimer such as an amino-terminated Starburst™ PAMAM dendrimer (Aldrich).
l) cationic acrylamide homo- or copolymers, and their modification products, such as poly(acrylamide-co-diallyldimethylammonium chloride) or glyoxal-acrylamide-resins;
m) polymers formed by polymerisation of N-(dialkylaminoalkyl)acrylamide monomers,
n) condensation products between dicyandiamides, formaldehyde and ammonium salts,
o) typical wet strength agents used in paper manufacture, such as urea-formaldehyde resins, melamine-formaldehyde resins, polyvinylamine, polyureide-formaldehyde resins, glyoxal-acrylamide resins and cationic materials obtained by the reaction of polyalkylene polyamines with polysaccharides such as starch and various natural gums, as well as 3-hydroxyazetidinium ion-containing resins, which are obtained by reacting nitrogen-containing compounds (e.g., ammonia, primary and secondary amine or N-containing polymers) with epichlorohydrine such as polyaminoamide-epichlorohydrine resins, polyamine-epichlorohydrine resins and aminopolymer-epichlorohydrine resins as for instance mentioned in U.S. Pat. No. 3,998,690.

Preferred polycationic polymers are cationic or cationically modified polysaccharides such as starch or cellulose derivatives, chitin, chitosan or alginate, polyallylamine homo- or copolymers, polyvinylamine homo- or copolymers or polyethylenemine.

According to one embodiment a positively charged polymer with hydrophobic side chains is particularly advantageous. The polymer can be applied by spraying, roll-coating or slot-coating from water solutions and then drying the solution. The bacteria will adhere to the fibers and stay in place. The migration of the bacteria will be reduced or prevented, since the bacteria are trapped in or on the first element or section, hence, the bacteria are immobilized onto the fibrous material. For trapping bacteria on the skin of the user, contact between the article and the skin of the perineum is required.

The immobilization effect can be affected by modifying the fibers with functional groups capable of interacting with the bacterial cell walls. Such functional groups include cationic groups and carbonyl groups, which can be introduced into the fibers by direct chemical modification of the fibrous material. Other examples of functional groups are ammonium groups, phosphonium groups, sulphonium groups and certain metal containing groups. These groups are electropositive. The functional groups may be the same throughout the fiber or the fiber can contain different functional groups.

The modified fiber can be a cellulosic fiber. Further, the fiber may also be synthetic, such as a polyester, polypropylene, polyvinyl alcohol, polyamide, polylactic acid and the like. The fibers can form some form of nonwoven or woven material.

The fibers could be arranged as a part, i.e., a section, of the top sheet or as a strip, i.e., an element, of the positively charged material and then arranged on the top sheet. Hence, the positively charged material is arranged in a first element or a section according to the present invention. A person skilled in the art will be able to select appropriate concentrations. EP 1 583 567 gives examples of appropriate concentrations of positive charges and of aldehyde functionalities.

The concentrations mentioned for the different materials depend on what kind of article it refers to. A large incontinence article may for example need a larger amount of hydrophobic material, antibacterial agent or positively charged material than a panty liner. The concentrations also vary depending on what material is used as the first element or section. When regarding the concentrations, a person skilled in the art could also determine which concentration would be needed to get the desired result.

We now refer to the design of the first element or section. The presence of bacteria, such as E. coli, in the urogenital area is dependent on the distance from the anus. The prevalence of E. coli is about 3 times higher in the perineum than in the labia majora. Therefore, it is very important to have a hinder, barrier or obstruction between the anus and the urogenital area. The distance between the anus and vagina is about 1 to 4 cm in different individuals. The size of the first elements or sections and is adapted to this measurement.

According to an embodiment of the invention, the distance between the side borders 126, 226 of the first element or section is at least 2 cm. This 2 cm distance corresponds quite well to the width of the perineum, on which the microorganisms can migrate. The first element 123, 223 or section having a distance of 2 cm between the side borders 126, 226, will work as a very good barrier when it is positioned tightly against the perineum of a wearer. The first element 123, 223 or section will reduce or prevent the migration of microorganisms through the perineum. The upper limit of the distance between the side borders 126, 226 of the first element 123, 223 or section, corresponds to the width of the microorganism hindering article or the absorbent article. Hence, the side borders 126, 226 of the first element 123, 223 or section can coincide with the longitudinal side edges 113, 114, 213, 214 of the article. The width of the article will vary depending on the article. An incontinence guard may for example be wider than a panty liner.

According to an embodiment, the distance between the front border 124, 224 and the rear border 125, 225 of the first element 123, 223 or section is at least 0.5 cm. The distance is suitable for preventing or reducing microbial migration. In a further embodiment, the distance between the front 124, 224 and rear 125, 225 borders could be at least 1 cm, or preferably at least 2 cm, depending on which microorganism hindering technique which is used in the first element or section. It also depends on which kind of article the first element or section is used in. The distance between the front border and the rear border can be as much as up to 4 cm in the microorganism hindering article. It can even be a distance of about 5 to 6 cm, since the first element or section can extend behind the perineum area of the user. According to one embodiment, the first element or section also could include or cover the whole top sheet of the microorganism hindering article. In the absorbent article, the distance between the front and rear borders of the first element or section could be up to 4 cm as in the microorganism hindering article. Further, the distance between the front and rear borders could be as much as about 6 cm. In such a case, the first element or section will extend back to the anus area during use, i.e., behind the perineum area.

The first element or section according to the invention provides a hinder, barrier or obstruction for microbial migration from the anus to the urogenital area of a wearer. The migration of microorganisms is prevented both on the microorganism hindering article or the absorbent article and on the skin of the wearer. It is very important to have close contact between the perineum skin of the wearer and the article. It is thereby ensured that a barrier to microorganism transport is formed. The migration on the perineum skin could be prevented by contact with the first element or section which inhibits microorganisms. Alternatively, the first element or section can transmit microorganism hindering material to the skin of the wearer. This also requires close contact between the article and the skin of the wearer.

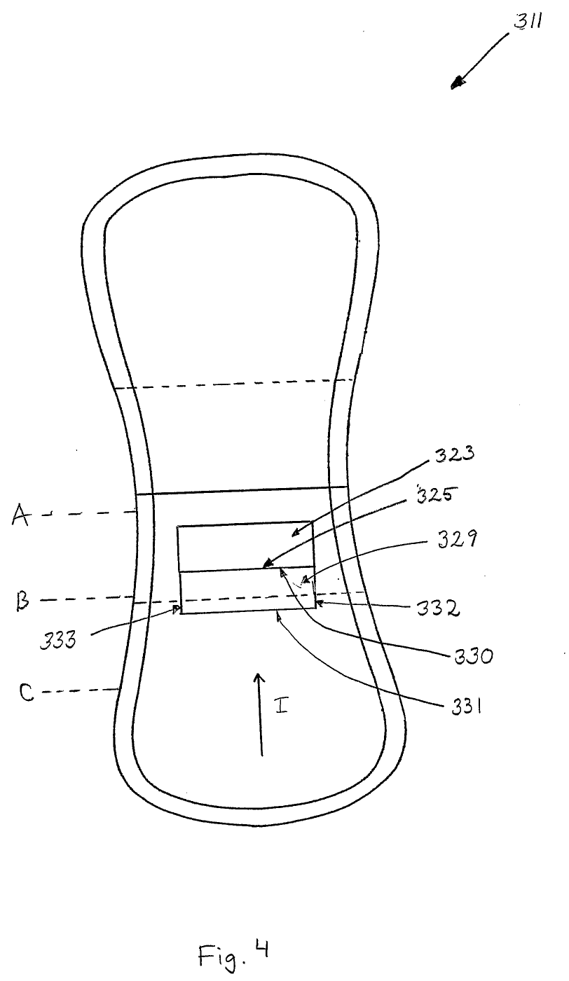

Another embodiment is shown in FIG. 4. This sanitary napkin corresponds partly with the sanitary napkin in FIG. 3, where certain parts of the FIG. 4 sanitary napkin are not shown. In addition to having a first microorganism hindering element 323 or section, the article 311 also includes a second microorganism hindering element 329 or section, having a front border 330 and rear border 331 and two side borders 332, 333, wherein the second element 329 or section extends backward from the rear border 325 of the first element 323 or section. The front border 330 of the second element 329 or section coincides in this case with the rear border 325 of the first element 323 or section, but this is not essential. The front border 330 of the second element 329 or section can be in front of or behind the rear border 325 of the first element 323 or section. Hence, the second element 329 or section can partly overlap the first element 323 or section. The points A, B and C are marked in the Figure and correspond to the points in FIG. 1 when the article is in use.

In an embodiment according to the invention, the article includes a first element 323 or section including a hydrophobic agent or a positively charged material and a second element 329 or section including an antimicrobial agent. The reason for arranging the antimicrobial agent in the second element 329 or section is that certain antimicrobial agents can disturb the normal flora in the urogenital area. Hence, it is better that such an agent is closer to the anus thereby reducing the risk for disturbing the microflora in the urogenital area. The antimicrobial agent will inhibit bacteria on the skin close to the anus. Smaller molecules, such as Iodophores or metal salts, are most appropriately situated in the second element 329 or section, because then they will be sufficiently far away from the urogenital area that migration to the urogenital area and subsequent disturbance of the vaginal flora will be minimal.

The second element or section will have the same dimensions as the first element or section, i.e., the distance between the front and rear borders is at least 0.5 cm, and in other embodiments the distance is at least 1.0 cm or at least 2.0 cm. The distance between the side borders is at least 2 cm.

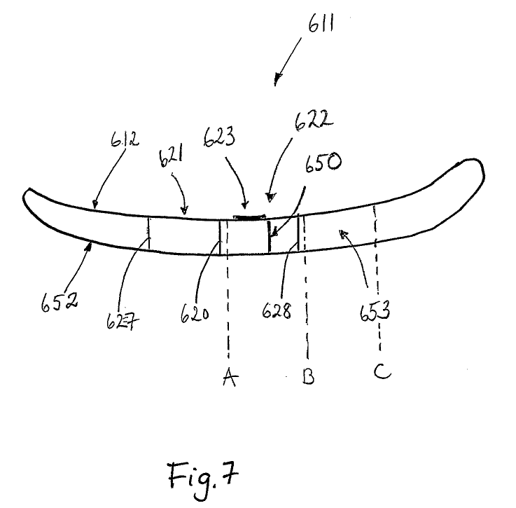

As shown in FIG. 7, a third element 650 could also be arranged in the absorbent layer 653. The FIG. 7 embodiment is a sanitary napkin. The third element 650 is in the form of a film or a fibrous layer extending between the longitudinal edges of the absorbent article 611 and extending vertically between the top sheet 612 and the bottom sheet 652. A first element 623 or section is shown on the top sheet 612. Microorganisms from the anus can migrate through the absorbent layer 653 from the corresponding anus area to the corresponding urogenital area. To hinder this, a third element 650 situated in the absorbent layer 653 can protect the absorbent layer corresponding to the urogenital area from microbial contamination. The third element 650 could be a plastic film, such as a film of polyethylene, which prevents microorganisms from moving to the other side of the plastic film. Hence, there will be no transportation of liquid or microorganisms between the parts of the absorbent layer divided by the third element 650. The third element 650 could also be a fibrous layer including a positively charged material as mentioned above. The bacteria would then be attracted to the positively charged material as described above. The third element 650 could also be included of modified fibers, which could be positively charged fibers or fibers incorporating metal salts or triclosan. The fibers can be natural or synthetic. Further, the points A, B and C corresponding to FIG. 1 are shown and the central line 620 is also shown. The front border 627 and the rear border 628 of the crotch portion is also shown for illustrating the position of the first element 623 and the third element 650 in the article in FIG. 7. The crotch portion is also divided in a front part 621 and a rear part 622.

The article may include one or several of the first, second or third elements or sections according to the present invention.

It is important that the hindering element or section does not come into contact with the urogenital area of the user. This is especially important when the element or section includes an antimicrobial agent. The reason is the same as mentioned above, i.e., the natural flora in the urogenital area can be disturbed. The natural healthy vaginal flora is an important part of the defense against infection. Further, the absorption capacity of the article could be negatively affected if the material or agent covers a large portion of the top sheet of an absorbent article. However, there is a difference between the microorganism hindering article and the absorbent article. The microorganism hindering article does not necessary absorb liquid and it is small relative to the absorbent article. This means that it is easier to place the microorganism hindering article so that the first element or section will be situated against the perineum area and not at the urogenital area. Hence, the material or agent will not disturb the urogenital area. Therefore, the first element or section could include or cover the whole top sheet of the microorganism hindering article.

The element or section could be protected with a protective covering or film, such as a plastic film. This could for example prevent hydrophobic material from flowing out and protect the antimicrobial material from being transferred to other parts of the article. The element or section would also be protected from the environment if it is sensitive to moisture or air for some reason. The covering film should be removed prior to use.

The article according to the present invention can include attachment devices on the bottom sheet, on the side directed from the top sheet, in the form of adhesive. The adhesive is used for attachment to a user's underpants during use of the article. The adhesive may be a pressuresensitive hotmelt glue. However, it is possible to use other commercially available pressure-sensitive adhesives, and also adhesives that are pressure-sensitive in a cold state, such as acrylate glue, normally combined with additives that enhance the stickiness of the glue, such as polyterpene or hotmelt glue, such as styrene and butadiene co-polymers. The attachment devices can be such as those already known in the art. Further, they can be covered by protecting films. In the package state of the absorbent article, the adhesive devices are covered with a protective layer, e.g. with release paper including a silicone-coated paper and functioning to protect the adhesive devices against contaminants, such as dust and similar substances, and also to prevent the adhesive from drying out prior to use. The absorbent article is conveniently provided with a central protective layer which is not removed until the absorbent article shall be fastened to the inside of underpants, and a protective layer on the adhesive, the protective layer being removed prior to positioning the article in the underpants.

The microorganism hindering article could as mentioned be attached to underpants or clothing of the wearer or to the wearer. If it is attached to the wearer, it is intended to be placed against the perineum. The woman using the article can feel how to place it, since it is easy to know where the perineum starts, just behind the vagina. The article may then include attachment devices on the top sheet in the form of adhesive or a sticky agent covering the whole or parts of the top sheet. The sticky agent could be applied in stripes or points on the top sheet. The sticky agent can include polysaccharides, for example pectin. Another example of an agent to use as a sticky agent is alginate. In order to avoid undesired growth of microorganisms in the sticky agent, it should have a pH below 5. This could be achieved by addition of a pH lowering agent in the form of an acid. Alternatively an antimicrobial agent such as chitosan or peroxides can be added. Silicone gel could also be used as an adhesive for attachment, either on the edges or on part of the top sheet. The attachment to the skin of the wearer described above could also be used with an absorbent article, if it is a panty liner. However, a panty liner is preferably provided with some kind of marking for a correct positioning against the perineum.

The microorganism hindering article could be in the form of a wadding. The wadding could have a hydrophobic material, an antimicrobial agent incorporated in it or it could be coated with such material or agent. Further, the wadding could be formed of or coated with positively charged material, such as modified fibers as described above.

The invention also relates to an embodiment wherein the article includes lactic acid producing bacteria in at least a part of area which contacts the urogenital region of the wearer during use. This part of area which includes lactic acid producing bacteria is in front of the central line of the crotch portion. Further, the invention also relates to an embodiment wherein the article includes lactic acid bacteria in the front part of the crotch portion. These bacteria strengthen the user's own defense system by contributing to a positive microflora, while the first element or section at the same time prevents or reduces migration of microorganisms from the anus to the urogenital tract.

The articles and the products could be packaged in conventional packaging. It is important that the packaging is water and moisture impermeable if the element or section is sensitive to moisture.

If it is not clear what part of the absorbent article is the front part, it should be explained to the user. For example, it could be shown on the article. This could be a simple mark which shows the front or rear part. Otherwise, the hindering element or section could be marked by some kind of marking or coloring. It is preferred that the first element or section is marked, and this can have another purpose according to the present invention as will be described below.

As earlier described, it is very important that the microorganism hindering article or absorbent article is positioned in the correct place on the wearer. The first microorganism hindering element or section should be placed against the perineum since it is in this place the element or section should be active. Further, it is important that when the article is used, the element or section does not come in contact with the urogenital area, since this could be disruptive for the normal flora in the urogenital area. For aligning the first element or section of the article against the perineum in the right way on the wearer, there may be provided a marking in the form of a picture or a coloring on the top sheet where the first element 123 or section is provided. If the form of article does not automatically ensure correct placement, marking may be needed. With reference to the figures, these markings and coloring would coincide with the first elements or sections. When positioning the article, the woman can put a finger on the bottom sheet just under the marking or coloring. Then, the woman needs to align the finger with the perineum having the article between the finger and the perineum. If more than one element or section is used, the first element or section should be aligned with the perineum. According to another embodiment, marking in the form of a picture or a coloring can be provided on the bottom sheet, on the side facing away from the user, on the place corresponding to the first element 123 or section on the top sheet, in order to align the first element 123 or section of the article at the perineum of the wearer. In such an embodiment the woman can also put her finger on the marking, but on the same side as the marking is situated, and then align the finger with the perineum with the article there between. The marking could also be used on the microorgansim hindering article.

The marking could also be tactile. The marking could for example have a rough, knotty or curved surface, which helps the user keep their finger on the marking while positioning the article so that the first element or section is aligned with the perineum of the wearer. The marking could also be in the form of a shallow cavity designed to receive the finger.

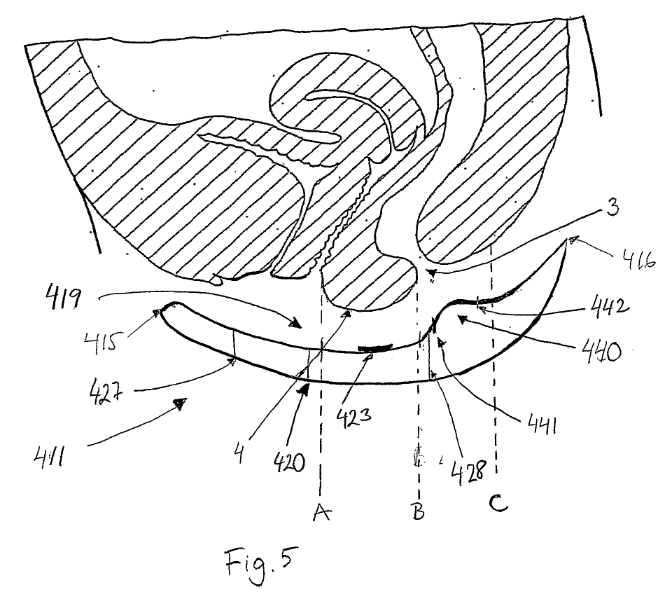

Referring to the absorbent article in the form of a sanitary napkin and FIG. 5, the sanitary napkin is shown together with an anatomic cross-sectional view of a human female illustrating the genitals. According to one embodiment of the invention, the article 411 further includes an anal hump 440 in the rear portion, wherein the anal hump has a front limit 441 and a rear limit 442 and two side limits, not shown. The anal hump 440 is intended to fit against the anus 3 of the wearer during use in order to align the first element 423 or section with the perineum 4 of the wearer during use, wherein the distance between the front limit 441 of the anal hump 440 and the center line 420 of the crotch portion 421 is at least about 1 cm and the distance between the front 441 and the rear 442 limits of the anal hump 440 is at least about 0.5 cm. The anal hump will have a height of at least 0.5 cm compared to the base level of the article, wherein the base level here corresponds to the level of the top sheet for a plane article. Humps are commonly known and the skilled person would know how to construe a hump, but here the position is crucial according to the present invention. The crotch portion 419 is situated between the front border 427 and the rear border 428 shown in the Figure.

As mentioned above, the purpose with the anal hump is to fit against the anus. This in turn achieves the goal of placing the first element or section against the perineum during use. The woman using the absorbent article can feel if the article is not fitted correctly, since the article will be uncomfortable to wear if the anal hump is not fitted against the anus. This embodiment ensures that a migration of microorganisms on the skin in the direction from the anus to the urogenital region will be prevented or reduced. The construction of the absorbent article will ensure the contact with the first element or section against the perineum of the wearer. The migration on the skin is therefore prevented.

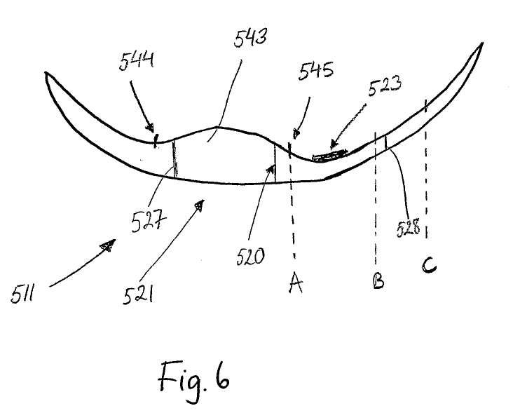

As another embodiment in the form of a sanitary napkin, shown in FIG. 6, the article 511 includes a vaginal hump 543 having a front limit 544 and a rear limit 545 and two side limits. The vaginal hump 543 is intended to fit against the vagina of the wearer during use, in order to align the first element 523 or section with the perineum of the wearer during use, wherein the rear limit 545 of the vaginal hump 543 is situated about 0.5 cm in front of to 0.5 cm behind the center line 520 of the crotch portion 521 and the distance between the rear limit 545 and the front limit 544 of the vaginal hump 543 is at least about 0.5 cm. The distance between the rear limit 545 and the front limit 544 can be about 0.5 to 10 cm, and is according to one embodiment 0.5 to 3 cm. The height of the hump is about 0.3 to 2 cm. The distance between the side limits is about 0.5 to 2 cm.

The purpose with the vaginal hump is to fit against the vagina. This in turn ensures that the first element or section will be placed against the perineum during use. The woman using the absorbent article can feel if the article is not fitted correctly, since the article will be uncomfortable to wear if the vaginal hump is not fitted against the vagina.

The article can also include two humps, one fitting against the vagina and one fitting against the anus.

As mentioned earlier, the crotch portion is intended to be placed against the crotch of the user. The crotch portion will be situated differently in the article depending how the absorbent article is constructed. If the absorbent article for example is symmetrical, such as in FIG. 2, the center line 120 of the crotch portion is situated in the middle of the absorbent article. The middle of the article is easily found by folding the front and rear edges of the article against each other.

Further, if the absorbent article has an hour glass form, the center line 120 of the crotch part 121 is situated where the distance between the longitudinal side edges of the article is the smallest.

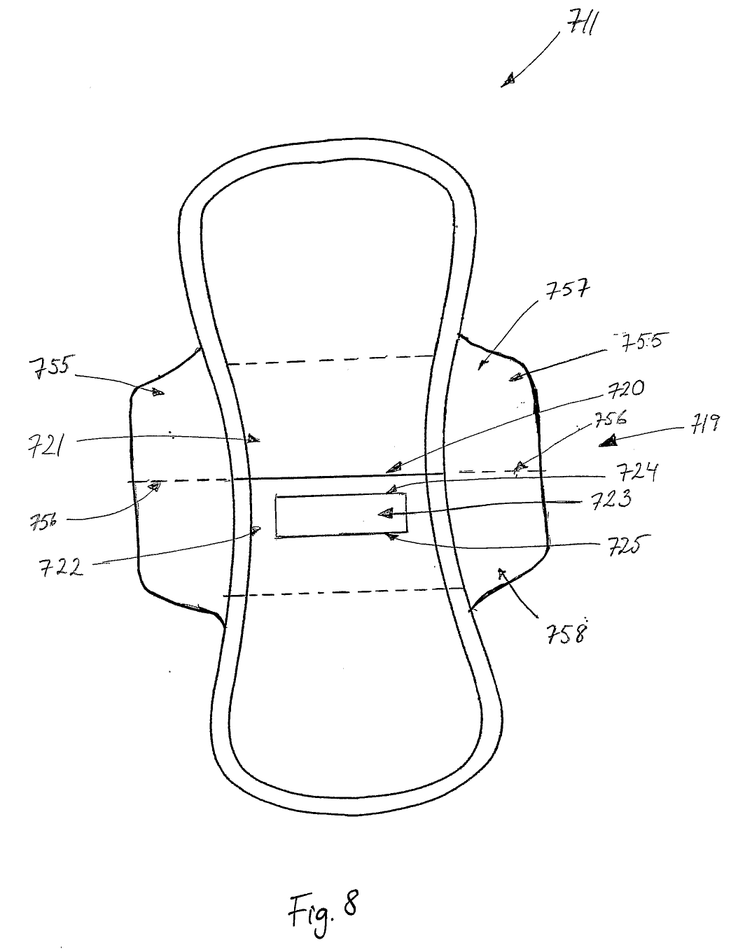

In FIG. 8 a sanitary napkin 711 having wings 755 is shown, where the wings 755 are used for folding around the crotch of underpants during use. The wings will keep the sanitary napkin on the right place in the underpants, and the underpants are protected from body liquid, since the wings will protect the liquid from leaking out at the edges and reaching the underpants. A crotch portion 719 is shown having a front part 721 and a rear part 722. A first microorganism hindering element 723 or section, having a front border 724 and a rear border 725, is arranged so that the front border 724 is situated in the rear part 722 of the crotch portion 719. The front border 724 of the first element 723 or section is situated about 0.5 to 3 cm from the center line 720 of the crotch portion 719. In this embodiment, the wings will help to align the first element or section to the perineum. The crotch part 719 of the absorbent article will be situated between the wings 755. The wings are divided in a front part 757 and a second part 758, divided by a center line 756. When an absorbent article including wings is used, the central lines 756 of the wings 755 coincide approximately with the central line 720 of the crotch part of the absorbent article or the central line 720 of the crotch portion 719 will be situated about up to about 1 cm forward of the central line of the wings. The wings 755 will be aligned to the crotch part of the underpants, which means that the crotch portion 719 of the absorbent article will be aligned to the crotch of the wearer. This ensures that the first microorganism hindering element 723 or section will be aligned to the perineum and thereby a correct placement is ensured.

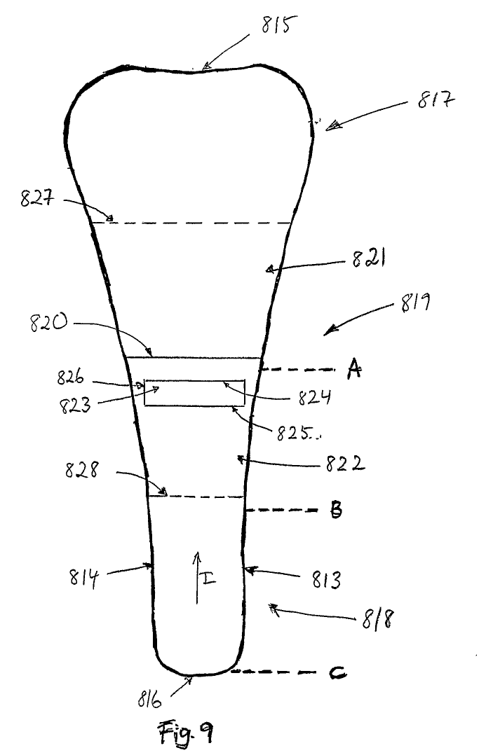

In FIG. 9 an embodiment in the form of a panty liner for thong-style underwear is shown. The article includes two essentially longitudinal side edges 813, 814, one essentially transverse front edge 815 and one essentially transverse rear edge 816. It includes a front portion 817, a rear portion 818 and a crotch portion 819 which is located between the front 817 and rear 818 portions. The crotch portion is divided in a front part 821 and a rear part 822 by a central line 820. The article includes at least one first microorganism hindering element 823 or section, having a front border 824 and a rear 825 border and two side borders 826, the first element 823 or section intended to be placed against the perineum of the wearer during use. The first element 823 or section is arranged so that the front border 824 of the first element 823 or section is situated in the rear part of the crotch portion about 0.5 to 3 cm behind the center line 820 of the crotch portion.

It is, as mentioned earlier, important that good contact is achieved between the first element or section of the absorbent article and the skin of the wearer at the perineum. It is also important that contact remains good in order to maintain the barrier to microorgansim transport during use of the product. Therefore, it is advantageous if the absorbent article or the micoorganism hindering article has a three-dimensional shape. If the article has a shape that is three-dimensional on the side directed against the user, it should have a shape, such that it is bulged especially in the part of the article which will be positioned in the area of the perineum of the user. That would lead to the article having constant contact with the perineum.

An example of a microorganism hindering article having a three-dimensional shape is a microorgansim hindering article including a fold in the longitudinal direction in the middle of the article, so that a bulge is achieved by the fold on the top sheet side of the article in the longitudinal direction.

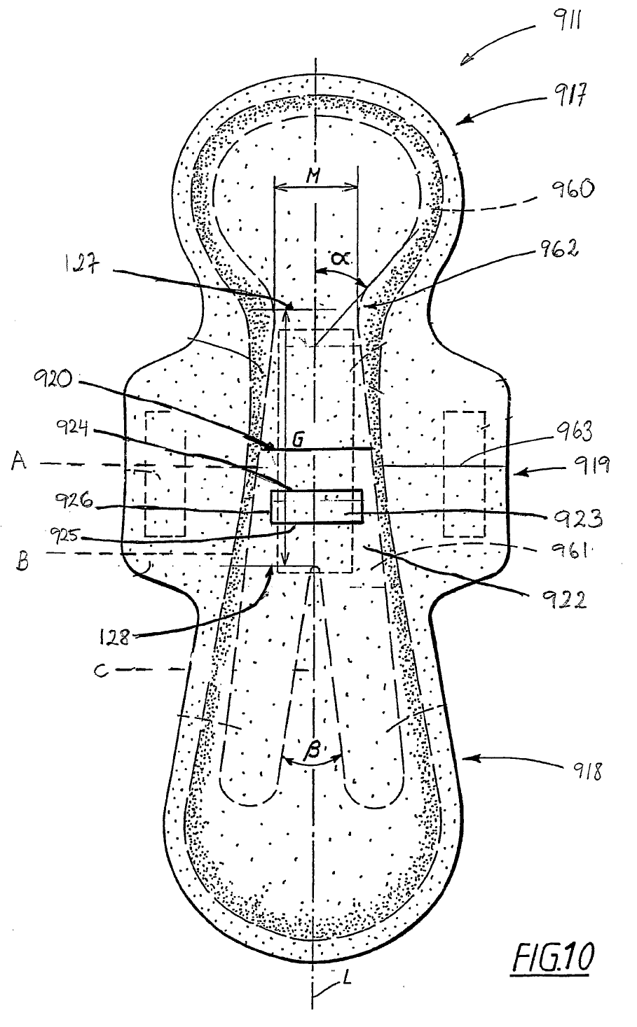

Further, an embodiment of an absorbent article according to the present invention having a three-dimensional shape is shown in FIG. 10. It discloses an absorbent article, which article has a longitudinal direction and a transverse direction, a front portion 917, a rear portion 918, a crotch portion 919 located between the rear portion and the front portion, an absorbent layer and a liquidtight layer 960 and also a stiffening element 961 which is intended to contribute to the three-dimensional shape of the article during its use. The stiffening element 961 is in a plane state before use of the article, it extends in the longitudinal direction of the article over the crotch portion 919 and at least some way in over the front portion 917. Further, the stiffening element 961 has a width M at the transition 962 between the crotch portion 919 and the front portion 917 which is adapted to the distance between the muscle tendons gracilis of the wearer on both sides of the crotch of the wearer in the groin of the latter and which is about 2.5-4.5 cm and the crotch portion 919 has a length G of about 7-12 cm. The side edges of the stiffening element 961 in the front portion 917 of the product diverge in the direction from the crotch portion 919 at least some way in over the front portion 917 and the side edges of the stiffening element 961, in the direction from the crotch area, form an acute angle α with a line in the longitudinal direction of the product. Further, the article includes a first microorgansim hindering element 923 or section arranged so that the front border 924 of the first element 923 or section is situated in the rear part 922 of the crotch portion 919 about 0.5 to 3 cm behind the center line 920 of the crotch portion 919. Here, the center line 920 of the crotch portion will be situated at a point forward of the center line 963 of the wings.

The article is positioned in connection with it being put on with the transition between the front portion and the crotch portion located between the muscle tendons. It should be fixed in between the tendons and in this way be transformed from plane form to three-dimensional form with the front portion curved upwards in relation to the crotch portion and forming a bowl-like shape at least in an area next to the crotch portion. With this three-dimensional form, the first element or section will have a constant contact with the skin of the wearer and the microorganism barrier will remain during use of the article. The migration of microorgansims on the skin of the perineum will thus be prevented or reduced.

During use of the article, the rear portion 918 is folded along the line L. Further embodiments of three-dimensional absorbent articles are disclosed in WO 02/087483, which is hereby incorporated by reference. All the embodiments described therein for female use could be used with the microorganism hindering element or section according to the present invention.

The stiffening element can include a dry-formed fiber mat with a density between 0.15 and 0.75 g/cm³ and a weight per unit area of about 100-400 g/m². It may include a mixture of cellulose fibers and viscose fibers, and it may also contain synthetic melt fibers.

The articles and the products according to the present invention could be packaged in conventional packaging. It is important that the packaging is water and moisture impermeable if the element or section is sensitive to moisture.

The microorganism hindering article described above could also be used together with an absorbent article, such as a sanitary napkin, a panty liner or an incontinence guard. Some women may need microorganism hindering articles between menstruations. Then during the menstruation, they will need the absorbent articles also. Many women regularly use panty liners or incontinence guards. It could then be convenient to use the microorganism hindering article together with an absorbent article.

Hence, the present invention further relates a kit for preventing or reducing migration of microorganisms from the anus area to the urogenital area including:
a. an absorbent article, such as a sanitary napkin, a panty liner or a incontinence guard, including a liquid-pervious top sheet intended to be directed against the wearer during use and a liquid-impervious bottom sheet intended to be directed against the underpants of the wearer during use, which article has a longitudinal direction, a transverse direction, two essentially longitudinal side edges, one essentially transverse front edge, one essentially transverse rear edge, a front portion, a rear portion, and a crotch portion located between the rear portion and the front portion, the crotch portion being divided in a front part and a rear part by a center line, the crotch portion having a length of about 7 to 12 cm, and the absorbent article having a marking on the top sheet intended for aligning a microorganism hindering article according to any one of claims 2 to 12 so that the first element or section will be placed against the perineum of the wearer during use for preventing or reducing migration of microorganisms in the direction from the rear edge to the front edge of the article, and
b. a microorganism hindering article according to any one of claims 2 to 12, wherein the microorganism hindering article is designed to be attached to the top sheet of the absorbent article so that the front border of the first element or section will be placed against the perineum of the wearer during use.

The same material and agents as described above are used, and the articles should be situated so that the first element or section will be placed against the perineum as described above.

When the microorganism hindering article is used for the kit together with an absorbent article, it could have attachment devices on the back sheet on the side intended to be directed from the wearer, i.e., the side intended to be attached against the absorbent article. The attachment devices could be in the form of adhesive on the edges of the back sheet, in the form of strips on the attachment devices or the whole back sheet could be covered with adhesive. The adhesive could be covered with a protective cover, which should be removed before use.

For some women it would be convenient to use a conventional absorbent article together with a composition for preventing or reducing migration of microorganisms which they apply to the absorbent article. Further, some antimicrobial agents could be sensitive to environmental influences and could be kept separate from the absorbent article.

This has been solved according to the invention by a kit for preventing or reducing migration of microorganisms from the anus area to the urogenital area during use including:
a. an absorbent article, such as a sanitary napkin, a panty liner or a incontinence guard, including a liquid-pervious top sheet intended to be directed against the wearer during use and a liquid-impervious bottom sheet intended to be directed against the underpants of the wearer during use, which article has a longitudinal direction, a transverse direction, two essentially longitudinal side edges, one essentially transverse front edge, one essentially transverse, rear edge, a front portion, a rear portion, and a crotch portion located between the rear portion and the front portion, the crotch portion being divided in a front part and a rear part by a central line, the crotch portion having a length of about 7 to 12 cm, and the absorbent article having a marking, the marking having a front border, a rear border and two side borders, wherein the marking is situated so that it will be placed against the perineum of the wearer during use, the marking being intended for applying a composition for preventing or reducing migration of microorganisms, and
b. a composition for preventing or reducing migration of microorganisms including a material selected from a hydrophobic agent and an antimicrobial agent or a mixture thereof, and
c. a means, such a cotton bud, brush, or peg, for applying the composition onto the marking on the top sheet of the absorbent article.

The composition can be selected from the hydrophobic material and antimicrobial agent described above, and the composition should be applied on the top sheet on the marking, wherein the marking should correspond to the place where a first element or section is arranged in the articles described above and the first element or section should be placed against the perineum of the wearer during use. The positioning of the sections, elements or markings as well as the materials in the sections or elements in the kits mentioned above, are intended to be the same as described regarding the absorbent articles and the microorganism hindering articles described above.

Through the use of the invention a close contact between the article and the perineum is achieved and a barrier is achieved and maintained.

According to the present invention, a small amount of microorganism hindering material, such as hydrophobic material, antimicrobial agent and positively charged material, can have a large effect. The whole article does not need to be treated and it nevertheless has a very good effect on preventing or reducing microorganisms from moving from the anus to the urogenital area. The small amount used is advantageous for the women wearing the article or product, since they do not have to be in contact with large amounts of added material and because the healthy urogenital flora is not disturbed. Further, costs can be reduced if the material does not have to be applied to the entire top sheet.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. An absorbent article (111) for female use, such as a sanitary napkin, a panty liner or an incontinence guard, comprising a liquid-pervious top sheet (112) intended to be directed against the wearer during use and a liquid-impervious bottom sheet intended to be directed against underpants during use, which article has a longitudinal direction, a transverse direction, two essentially longitudinal side edges (113, 114), one essentially transverse front edge (115), one essentially transverse rear edge (116), a front portion (117), a rear portion (118), and a crotch portion (119) located between the rear portion (118) and the front portion (117), the crotch portion (119) being divided in a front part (121) and a rear part (122) by a central line (120), the crotch portion (119) having a length of about 7 to 12 cm, characterized in that the article comprises at least one first microorganism hindering element (123) or section, having a front border (124) and rear border (125) and two side borders (126), the first element (123) or section situated so that it will be placed against the perineum of the wearer during use and thereby prevents or reduces migration of microorganisms in the direction from the rear edge (116) to the front edge (115) of the article and thereby prevents or reduces migration of microorganisms from the anus area to the urogenital area during use, the first element (123) being arranged on the top sheet (112) or the first section being incorporated in the top sheet (112).

2. A microorganism hindering article (211) for feminine use, characterized in that the article comprises a top sheet (212) intended to be directed against the wearer during use and optionally a liquid-impervious bottom sheet intended to be directed against the underpants of a user during use, which article (211) has a longitudinal direction, a transverse direction, two essentially longitudinal side edges (213, 214), one essentially transverse front edge (215), one essentially transverse rear edge (216), a front portion (217) and a rear portion (218), the article (211) comprises at least one first microorganism hindering element (223) or section, having a front border (224), a rear border (225) and two side borders (226), the first element (223) or section intended to be placed against the perineum of the wearer during use and thereby prevents or reduces migration of microorganisms in the direction from the rear edge (216) to the front edge (215) of the article and thereby prevents or reduces migration of microorganisms from the anus area to the urogenital area during use, the first element (223) being arranged on the top sheet (212) or the first section being incorporated in the top sheet (212).

3. The article according to any of the preceding claims, characterized in that the first element (123, 223) or section comprises at least one material selected from a hydrophobic material, an antimicrobial agent and a positively charged material.

4. The article according to any of the preceding claims characterized in that distance between the side borders (126, 226) of the first element (123, 223) or section is at least 2 cm.

5. The article according to any of the preceding claims characterized in that the distance between the front border (124, 224) and the rear border (125, 225) of the first element (123, 223) or section is at least 0.5 cm.

6. The article according to any of the preceding claims, characterized in that the article (311) comprises a second microorganism hindering element (329) or section, having a front border (330), a rear (331) border and two side borders (332, 333), wherein the second element (329) or section extends backward from the rear border of the first element (323) or section.

7. The article according to claim 6, characterized in that the first element (323) or section comprises a hydrophobic agent or a positively charged material and the second element (329) or section comprises an antimicrobial agent.

8. The article according to any of the preceding claims, characterized in that the bottom sheet comprises attachment devices on the side which faces the user's underpants during use, in the form of adhesive.

9. The article according to claim 2, characterized in that the distance between the transverse rear edge (216) and transverse front (215) edge is from 2 cm to 6 cm and the distance between the longitudinal side edges (213, 214) is from 3 to 5 cm.

10. The article according to claim 2 or 9, characterized in that the first element (223) or section is situated in the front portion (217) of the article (211).

11. The article according to any one of claims 2 or 9-10, characterized in that it comprises attachment devices on the top sheet in the form of an adhesive or a sticky agent covering the whole or parts of the top sheet, wherein the attachment devices are intended to attach the article to the skin of the wearer during use.

12. The article according to any one of claims 2 or 9-11, characterized in that it comprises a fold in the longitudinal direction in the middle of the article, so that a bulge is achieved by the fold on the top sheet side of the article.

13. The article according to claim 1, characterized in that the first element (123) or section is arranged so that the front border (124) of the first element (123) or section is situated in the rear part (122) of the crotch portion (119).

14. The first element (123) or section is situated about 0.5 to 3 cm behind the centre line (120) of the crotch portion (119).

15. The article according to any one of claims 1 or 13-14, characterized in that the article further comprises an absorbent layer between the top sheet and the bottom sheet.

16. The article according to claim 15, characterized in that a third microorganism hindering element (650) is arranged in the absorbent layer (653).

17. The article according to claim 16, characterized in that the third element (650) is a film or a fibrous layer extending between the longitudinal side edges of the absorbent article and vertically between the top sheet (612) and the bottom sheet (652).

18. The article according to any one of claims 1 or 13-17, characterized in that a marking is provided in the form of a picture or a colouring on the top sheet (112) where the first element (123) or section is provided, in order to align the first element (123) or section of the article at the perineum of the wearer.

19. The article according to claim 1 or 13-17, characterized in that there is provided a marking in the form of a picture or a colouring on the bottom sheet, on the side facing away from the user, on the place corresponding to the first element (123) or section on the top sheet (112), in order to align the first element (123) or section of the article at the perineum of the wearer.

20. The article according to any one of claims 1 or 13-19, characterized in that the article comprises lactic acid producing bacteria in at least a part of area which contacts the urogenital region of the wearer during use.

21. The article according to claim 20, characterized in that the part of area which comprises lactic acid producing bacteria is in front of the central line of the crotch portion.

22. A kit for preventing or reducing migration of microorganisms from the anus area to the urogenital area comprising:
a. an absorbent article, such as a sanitary napkin, a panty liner or a incontinence guard, comprising a liquid-pervious top sheet intended to be directed against the wearer during use and a liquid-impervious bottom sheet intended to be directed against the underpants of the wearer during use, which article has a longitudinal direction, a transverse direction, two essentially longitudinal side edges, one essentially transverse front edge, one essentially transverse rear edge, a front portion, a rear portion, and a crotch portion located between the rear portion and the front portion, the crotch portion being divided in a front part and a rear part by a centre line, the crotch portion having a length of about 7 to 12 cm, and the absorbent article having a marking on the top sheet intended for aligning a microorganism hindering article according to any one of claims 2 to 12 so that the first element or section will be placed against the perineum of the wearer during use for preventing or reducing migration of microorganisms in the direction from the rear edge to the front edge of the article, and
b. a microorganism hindering article according to any one of claims 2 to 12, wherein the microorganism hindering article is designed to be attached to the top sheet of the absorbent article so that the front border of the first element or section will be placed against the perineum of the wearer during use.

23. A kit for preventing or reducing migration of microorganisms from the anus area to the urogenital area during use comprising:
a. an absorbent article, such as a sanitary napkin, a panty liner or a incontinence guard, comprising a liquid-pervious top sheet intended to be directed against the wearer during use and a liquid-impervious bottom sheet intended to be directed against the underpants of the wearer during use, which article has a longitudinal direction, a transverse direction, two essentially longitudinal side edges, one essentially transverse front edge, one essentially transverse rear edge, a front portion, a rear portion, and a crotch portion located between the rear portion and the front portion, the crotch portion being divided in a front part and a rear part by a central line, the crotch portion having a length of about 7 to 12 cm, and the absorbent article having a marking, the marking having a front border, a rear border and two side borders, wherein marking is situated so that it will be placed against the perineum of the wearer during use, the marking being intended for applying a composition for preventing or reducing migration of microorganisms, and
b. a composition for preventing or reducing migration of microorganisms comprising a material selected from a hydrophobic agent and an antimicrobial agent or a mixture thereof, and
c. a means, such a cotton bud, brush, or peg, for applying the composition onto the marking on the top sheet of the absorbent article.
